(19) United States
(12) Patent Application Publication
(10) Pub. No.: US 2013/0109009 A1
(43) Pub. Date: May 2, 2013

(54) BIOLOGICAL SAMPLE PREPARATION

(71) Applicant: Dan W. Kessel, Alto, TX (US)

(72) Inventor: Dan W. Kessel, Alto, TX (US)

(21) Appl. No.: 13/627,123

(22) Filed: Sep. 26, 2012

Related U.S. Application Data
(62) Division of application No. 13/285,956, filed on Oct. 31, 2011.

(51) Int. Cl. C12Q 1/24; C12Q 1/70

(52) U.S. Cl. 435/5; 435/30

(57) ABSTRACT

A biological sample collection and preparation kit includes a container-closure combination and collector including a handle, screen, and probe. The screen includes tapered apertures for separating sample debris from sample constituents collected via floatation. The probe is combined with the handle and screen to provide a disposable collector for safe and convenient sample collection. A vibratory agitation head includes a series of wells to secure containers thereon for sample preparation. The agitation head and containers are keyed to position containers on the agitation head in a desired orientation. The containers, collectors, and agitator head can be combined with a vibrator, preparation liquid, transfer slides and a slide handling, implements into a self-contained mini-lab station sample preparation kit, e.g., for onsite veterinary diagnosis.

### CROSS-REFERENCE TO RELATED APPLICATIONS

This application is a divisional of U.S. Ser. No. 13/285,956 filed on Oct. 31, 2011 by inventor Dan W. Kessel entitled BIOLOGICAL SAMPLE PREPARATION, which is incorporated herein by reference.

### BACKGROUND

The present invention relates generally to collection and preparation of biological samples, and in particular to a sample preparation kit for pre-microscopy mixing and preparation, e.g., floatation separation of microbes and parasite ova from biological samples.

### BACKGROUND

Biological sample collection and preparation is a routine part of medical diagnostic procedures. For example, veterinarians regularly use collection of liquid or solid samples to diagnose illness through detection of the presence of infective microbes, parasites, parasite ova or other foreign materials, e.g., roundworms, hookworms, whipworms, protozoa and bacteria. Such sample collection and preparation can expose diagnosticians to saliva, blood, urine, feces, and animal tissues, raising safety and hygiene concerns. Similarly, sample preparation equipment is often cleaned and reused, risking cross-contamination of subsequent samples.

Existing methods of parasite detection involve numerous steps and procedures that are potentially hazardous and can lead to contamination of an unloved hand and contamination of equipment. For example, intestinal parasite diagnosis generally requires the processing of a fecal sample so that demonstration may be made of the adult parasite, a larval stage of the parasite, or more commonly, the egg stage (ova or oocyst) of the parasite. The egg stage is often the most commonly attainable and identifiable, able to be differentiated from other parasites, and thus treated most effectively.

The most broadly used current methods of intestinal illness diagnosis include microscopic examination of sample preparations using fecal flotation, fecal sedimentation and direct fecal smears. Direct fecal microscopy is often inaccurate unless the sample has a high concentration of parasites.

Direct microscopy is, however, the best way to detect certain single-cell parasites such as giardia, amoebae, balamidium, and even large bacteria pathogens like spirochetes. The other procedures of flotation and/or sedimentation, often with centrifugation, can be used to detect lower concentrations of parasites, allowing a patient to be treated before outright clinical disease may be apparent.

Typical ova floatation sample preparation requires a period of time to allow for sufficient dissolution of the sample, sedimentation of debris and filtration of the test fluid and floatation of the ova. This process may not be conducive to some situations where rapid diagnoses are desirable.

Accordingly, improvements are sought in the safety, integrity, efficiency, cost-effectiveness and simplicity of biological sample collection and preparation.

### SUMMARY

It has been discovered that biological sample collection and preparation can be improved through using a novel disposable sample preparation kit and vibration offering greater safety, hygiene, efficiency and cost effectiveness.

Applicants have determined through experimentation that vibration of floatation samples and other biological samples can provide similar sample preparation results as centrifugation. Vibration of sorts, accelerates mobility allowing the various particles in the suspension to differ in position and orientation and allowing the ova to separate from debris and pass through apertures of a filter. Vibration also prevents sample debris from blocking filter apertures and allows passage of ova therethrough during floatation processes. The subject device and method improve upon direct microscopy, still flotation and centrifugation methods, e.g., in terms of hygiene, time efficiency, and accuracy.

One embodiment of a sample preparation kit is configured as a mini-lab station for multiple, processes e.g., separation and visualization, to be performed in the diagnosis of intestinal parasitism or other patient conditions.

In the case of fecal parasites, a small amount of feces is collected, using a probe, e.g., a fecal loop. This probe can be designed as part of a mixing tube top so that when fecal material is collected, the probe is placed in the tube, to which warm saline has been added if a preliminary direct exam is to he performed, or to which flotation or sedimentation fluid is added for respective procedures.

The tube is then placed into the top of the vibrator head and the vibrator is operated for a desired period, e one minute for direct microscopy and 3-5 minutes for flotation. This preparation process mitigates environmental and technician contamination and renders contaminated parts disposable as single-use items.

One aspect of the invention features, in some embodiments, a sample preparation kit suitable for onsite sample collection and preparation, e.g., during in-field diagnosis and treatment in some embodiments, a biological sample preparation kit includes a container for receiving a biological sample, a handle defining an exterior grip portion and an interior recessed screen portion and an elongated probe extending from the handle and configured to collect a biological sample. The handle is removably couplable to the container with the elongated probe received within the container. A vibratory agitator bead defines one or more wells configured to retain the container therein during vibratory agitation of the biological sample and liquid. The sample containers and vibratory agitator head are placed on a vibrator to expedite sample mixture and ova floatation through vibratory agitation.

In some implementations, the container and well are jointly keyed to position the container in one or more discrete container rotational positions within the well.

In some implementations, multiple wells are defined on the vibratory agitator head. The wells are keyed to accommodate a plurality of containers with respective hinged closures in a fully open position without interference with access to an adjacent one of the plurality of containers. In some implementations, the joint keying positions the container such that a hinged container closure opens outward relative to the vibratory agitator head. In some implementations, the keying comprises interference between one or more projections on the container and the well in all but the one or more discrete rotational positions.

In some implementations, the container comprises a plurality of radial projections defining a base about a tapered body portion of the container. In some implementations, the containers and/or the wells define a self-centering taper for self-centering of the container therein.

In some implementations, a sidewall portion of the well is splayable to provide a friction fit with a tapered body portion of a container received therein.

In some implementations, a sample preparation kit further includes a plurality of containers, a plurality of handles, a plurality of elongate probes, a plurality of glass slides, and one or more liquid reservoirs, e.g., containing sodium nitrate, zinc sulfate, or sugar solutions.

In some implementations, a sample preparation kit further includes a vacuum pen for indirect handling of the glass slides.

Another aspect of the invention features, in some embodiments, a biological sample preparation kit including a container for receiving a biological sample, a handle defining an exterior grip portion and an interior recessed screen portion and an elongated probe attachable to the handle to extend therefrom to collect a biological sample. The handle is removably couplable to the container with the elongated probe received within the container.

In some embodiments, the elongated probe is assembled at one end to the handle. In some embodiments, the elongated probe is secured within a recess in the handle via at least one of a snap-fit, interference fit, and threaded fit.

In some embodiments, the handle further defines a spillover recess between a handle grip and the screen.

In some embodiments, the screen defines a plurality of apertures therethrough with a plurality of the apertures being downwardly tapered.

Another aspect of the invention features, in some applications, a method of preparing a biological sample. The method includes manipulating a handle defining an exterior grip portion and an interior recessed screen portion to collect a biological sample at a distal end of an elongated probe extending from the handle. The method includes removably coupling the handle to a container with the elongated probe received within the container. The method includes positioning the container on a vibratory agitator head defining one or more wells configured to retain the container therein during vibratory agitation of the biological sample and agitating the biological sample within the container to aid floatation of a constituent of the biological sample to a fluid level above the screen.

In some applications, the method includes filling, of the container to a desired fluid level to float the constituent of the biological sample to an upper rim of the handle or the container; and transferring the constituent of the biological sample away from the fluid in the container via contact with an underside of a sample transfer device.

In some sample preparations, a small volume solution is prepared to detect motile flagellates and ciliates, like giardia, balamidium and also to detect some yeasts and coccidia, and spirochetes that may not readily float in solution. Preparation of biological samples can be performed with any combination of water, saline, stain or other agent.

In some applications, the transferring comprises handling the sample transfer device with a vacuum pen.

In some applications, positioning the container in the well further comprises aligning a keying feature of the container with a complementary keying feature of the well.

The detailed description refers to the accompanying drawings that show, by way of illustration, specific aspects and implementations in which the present disclosed teaching may be practiced. Other arrangements and implementations may also be utilized, and structural changes may be made without departing from the scope of the disclosed implementations. The various implementations are not necessarily mutually exclusive, as some implementations can be combined with one or more other implementations to form new implementations.

### BRIEF DESCRIPTION OF THE DRAWINGS

The present invention may be better understood, and its numerous objects, features, and advantages made apparent to those skilled in the art by referencing the accompanying drawings.

FIG. 1A is a perspective view of one embodiment of a biological sample preparation kit.

FIG. 1B is another perspective view of one embodiment of a biological sample preparation kit.

FIG. 1C is an exploded perspective view of one embodiment of a biological sample preparation kit.

FIG. 2A is a perspective view of one embodiment of a handle for biological sample collection probe.

FIG. 2B is a side view of the handle for a biological sample collection probe of FIG. 2A.

FIG. 2C is a top view of the handle for a biological sample collection probe of FIG. 2A.

FIG. 2D is a cross-sectional view of the handle for a biological sample collection probe of FIG. 2C.

FIG. 2E is a cross-sectional view of the handle for a biological sample collection probe of FIG. 2C.

FIG. 2F is a detailed view of the cross-sectional view of the handle for a biological sample collection probe of FIG. 2E.

FIG. 3A is a perspective view of one embodiment of a biological sample preparation container.

FIG. 3B is a side view of the container of FIG. 3A.

FIG. 3C is another side view of the container of FIG. 3A.

FIG. 3D is a top view of the container of FIG. 3A.

FIG. 4A is a perspective view of a vibratory agitator head for use with a biological sample preparation kit.

FIG. 4B is another perspective view of a vibratory agitator head for use with a biological sample preparation kit.

FIGS. 4C-4D are side views of the vibratory agitator head of FIG. 4A.

FIGS. 4E-4F are top views of the vibratory agitator head of FIG. 4A.

FIG. 4G is a cross-sectional view of the vibratory agitator head of FIG. 4F.

FIG. 5 is a perspective view of one embodiment of a biological sample preparation kit.

FIG. 6 is another perspective view of one embodiment of a biological sample preparation kit.

The use of the same reference symbols in different drawings indicates similar or identical items.

### DESCRIPTION OF THE PREFERRED EMBODIMENT(S)

Various sample preparation methods may be used to prepare and examine biological samples, e.g., blood, saliva, urine, feces, skin or muscle tissue, viral, fungal or bacterial specimens, hair, soil and plant tissues, or other organic materials, or foreign materials.

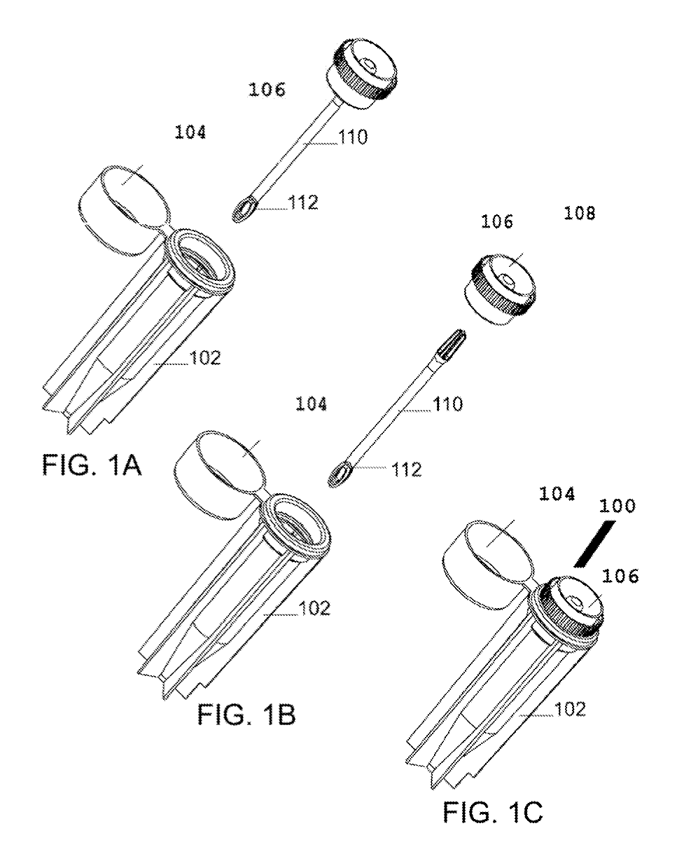

With reference to FIGS. 1A-1C, one embodiment of a biological sample preparation kit 100 includes a container 102 and closure 104 combination for preparing, storing, and transporting collected samples Kit 100 further includes a collector 106 having a handle 108 including an exterior grip portion and an interior screen or filter portion (not shown in these views). A probe 110 extends from handle 108 to provide a collector end 112 at a distance from handle 108.

Probe 110 can be assembled to handle 108, e.g., via snap-fit or interference-fit to form collector 1.06. In some implementations, probe 110 can be made integral with handle 108. Probe 110 can be removably attachable to handle 108, to facilitate interchange of probe types or sizes. Alternatively, probe 110 can be made to be non-removably attachable, e.g., by a looking snap-fit for fully disposable use.

Handle 108 can be received entirely within the container closure combination or may be positioned intermediate container 102 and closure 104 in a closed configuration.

In some embodiments, as depicted, probe 110 can be in the form of a loop. Still, in some embodiments, probe 110 can include any number or combination of collection features, e.g., bristles, scoop, absorbent swab, prongs, recesses, pipettes, scrapers, notches, serrations and the like. The probe may be sized to retrieve or collect a desired sample size and type.

The elongated probe can be rigid or semi-rigid depending on the desired application. For example, for stool collection, a substantially rigid, probe may be preferable, while a more pliable probe may be preferable for saliva collection.

In the example of feces collection and preparation, a tapered loop-styled probe facilitates ease of insertion, collection and removal of a stool sample. Probe 110 is then placed with the collected sample into reservoir 102 for further preparation. A feature within the container may be used to dislodge the sample from the loop. A desired amount of preparation liquid can then be added to the reservoir to dilute, dissolve, preserve, or react with the sample or achieve a particular sample consistency to prepare the sample for analysis.

In some cases, sufficient liquid is added to largely dissolve the sample and to fill the container above the level of the screen to allow for sedimentation and filtration of sample debris while allowing for floatation of ova within the sample.

Following floatation and sedimentation, to glass slide or other sample transfer device can then be used to isolate the ova or other target sample constituent from the container for further analysis.

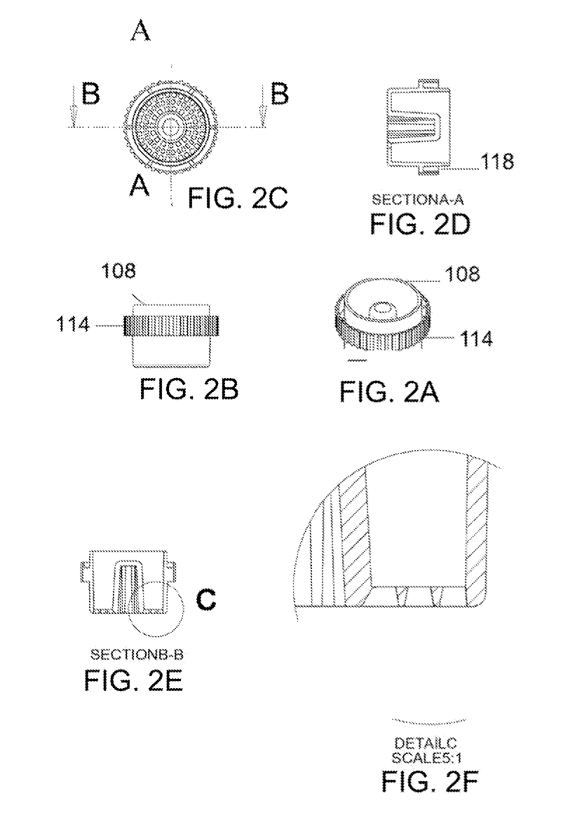

With reference to FIGS. 2A-2F, handle 106 includes an exterior grip portion 114 and an interior screen 114 extending across a major portion of the interior area of handle 106. A plurality of apertures or perforations define by screen 114 serve to prevent larger debris from floating to the surface of the preparation liquid in the closure. In some applications, vibration agitation or centrifugation may be used to expedite separation of sample debris from targeted floating particulate.

Screen 114 can be formed of any material and in any number of shapes suitable to serve as a sieve or filter. A series of apertures or perforations of screen 114 allow liquid to flow therethrough to separate particulate below from ova or other floatation particulate above. In some embodiments, and with reference to FIG. 2E-2F, the webs defining the apertures can be tapered downward to minimize the surface area presented to debris floating up against screen 114. Experimentation has shown that this tapered configuration can increase the number of ova that pass through screen 114 without substantially increasing the amount of large debris that passes through screen 114.

In some embodiments, an annular lip or rim provides an anti-spill feature 118 between an interior portion of handle 106 and the exterior grip 114 to mitigate contact with any spilled preparation fluid and sample material in the event of overfilling or minor spillage. Anti-spill feature 118 can include drainage passages back into container 102.

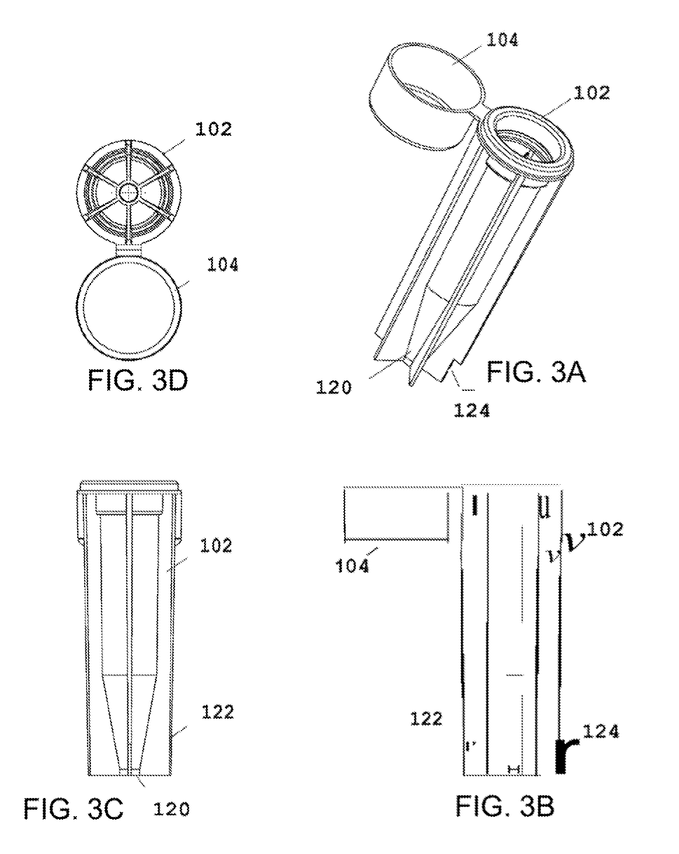

With reference to FIGS. 3A-3D, container includes sidewalk defining an interior reservoir. Closure 104 can be attached to container 102 or fully separable and can be closed thereon via snap-fit, interference-fit, threaded engagement, and the like. Closure 104 can include a lift tab to facilitate dislodging of closure 104 from container 102. Thus, closure 104 can be configured to be manipulated with one hand, e.g., through a tabbed flip-top and living hinge configuration. In some embodiments, a threaded closure 104 to container 102 interface may be manipulated with two hands.

Closure 104 can be preferably secured to container 102 to be leak free for storage, disposal or transportation, e.g., for delivery to a central laboratory, or for agitation, centrifugation, or other mechanical handling.

Container 102 and closure 104 can be formed of plastic, glass, metal or other materials suitable for preparation, storage and disposal of biological samples. In some embodiments, container 102, closure 104 and/or handle 106 can be configured with a spout, nozzle, line-coupler or other sample transfer feature.

Container 102 includes a tapered lower portion 120 that serves to provide a sample stirring, recess on the interior and on the exterior as a self centering feature for interfacing an agitator head. For example, in some cases, the tapered well at the lower portion of container body can be used to prepare a small volume suspension. A series of radial fins 122 extend outwardly from the tapered lower portion 120 to provide a stable base. In some embodiments, one or more radial fins 122 can include a keying feature 124 configured to align or otherwise position container 102 relative to a stand, agitator, storage or shipping container, and the like.

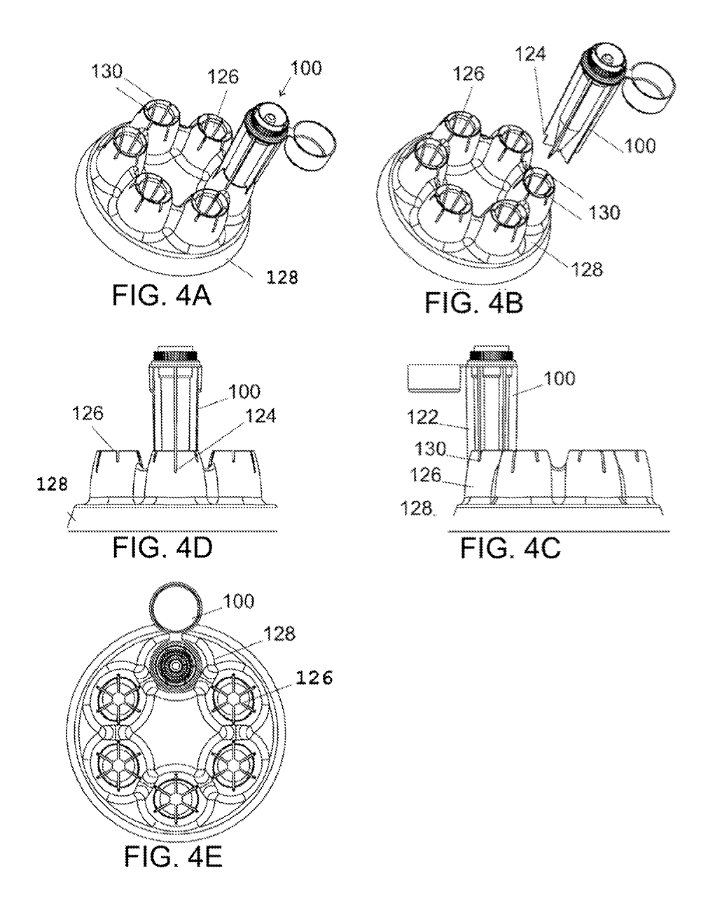

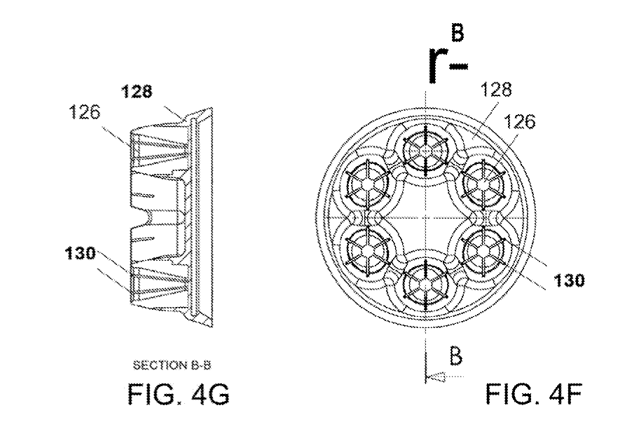

With reference to FIGS. 4A-4G, container 102 is received within a well 126 defined in a vibratory agitator head 128. Vibratory agitator head 128 is depicted as a rubber boot positionable on an active surface of a vibrator. Any number of other devices may similarly employ aspects and features of the invention. Head 128 is depicted as including a series of wells 126 for receiving the tapered base 120 of container 102 and including slits 128 for receiving fins 122 of container 102. Slits 128 can also allow sidewalls of wells 126 to be splayable to provide an interference fit to secure containers 102 therein during agitation.

One or more of slits 128 can be keyed to complement keying feature 124 on container 102 to position container 102 in a desired orientation. For example, when agitating multiple containers 102 on head 128, it may be desirable to ensure that closures 104 open outwardly on container 102 relative to head 128 to allow full access to each of containers 102. Full access can include clearance to permit a glass slide to be set atop container 102 to transfer ova floating on the surface of the preparation liquid in container 102.

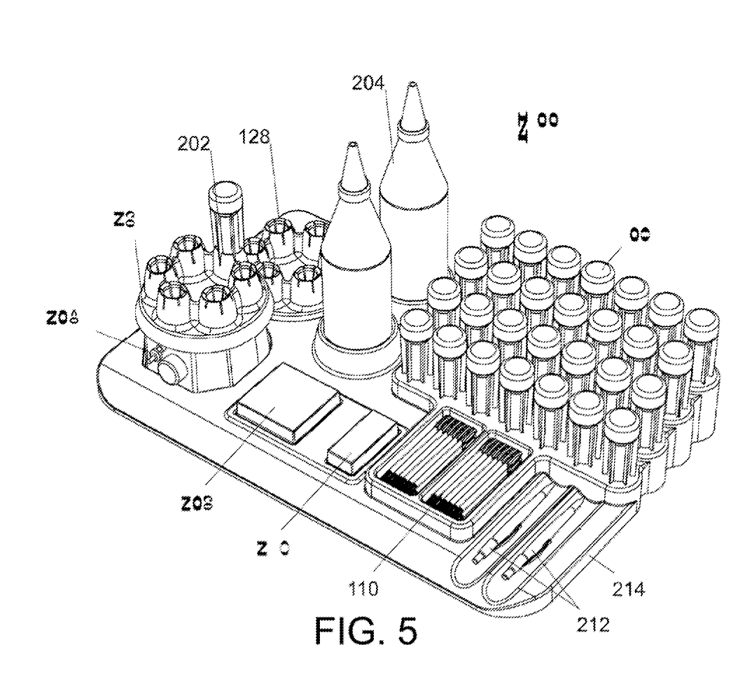

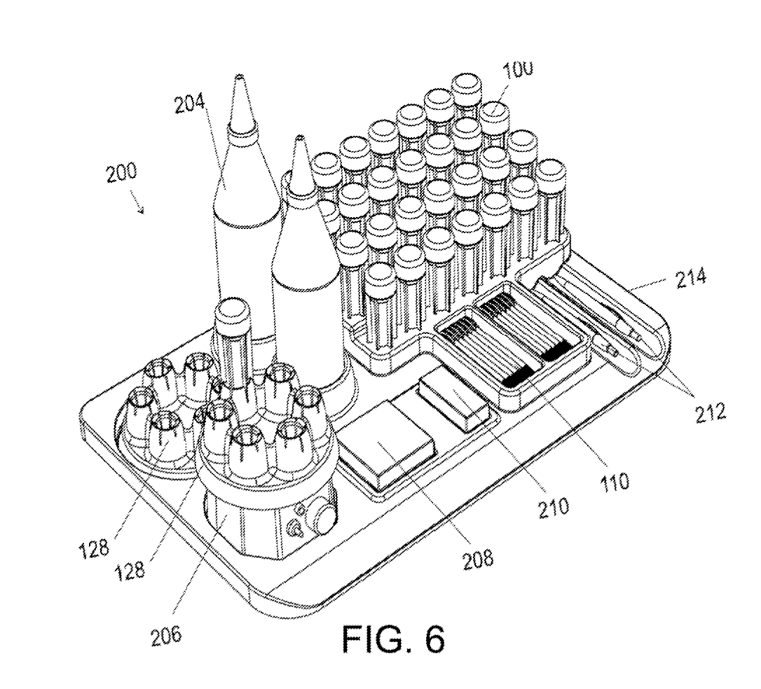

With reference to FIGS. 5-6, a self-contained biological sample kit 200 including a plurality of container-closure combinations 100 with handles 106 preassembled therein (not shown). A plurality of probes 110 are provided for further assembly with handles 106. Probes 110 can be of various sizes and forms for a wide range of collection and preparation scenarios.

Kit 200 further includes one or more preparation liquid reservoirs 204 for ready preparation of collected samples. A vibratory agitator 206 and one or more vibratory agitator heads 128 offer greatly accelerated dissolution of samples and floatation of ova. For example, experimentation has shown that vibration of sample preparations can significantly reduce the time required to achieve sufficient ova floatation and can greatly increase the number of collected ova.

A plurality of transfer slides 208 and 210 are provided for ready transfer of target floatation specimens. One or more suction pens 212, or other slide handling device, are provided for indirect handling of slides 208, 210 by technicians. A kit base 214 can include a plurality of wells or other dedicated regions for the various kit components for convenient access, presentation, storage and transport.

In some cases, tamper prevention measures may be provided for secure storage and shipping to preserve sample integrity. Such tamper prevention measures can include tape, shrink wrap, seals, printed cross-closure stickers and the like.

While the forgoing represents a description of various implementations of the invention, it is to be understood that the claims below recite the features of the present invention, and that other implementations, not specifically described hereinabove, fall within the scope of the present invention.

1-20. (canceled)

21. A biological sample preparation kit comprising:
a container for receiving a biological sample and a liquid;
a handle defining an exterior grip portion and an interior recessed screen portion;
an elongated probe extending from the handle and configured to collect a biological sample;
wherein the handle is removably couplable to the container with the elongated probe received within the container; and
a vibratory agitator head defining one or more wells configured to retain the container therein during vibratory agitation of the biological sample and liquid

22. The biological sample preparation kit of claim 21, wherein the container and well are jointly keyed to position the container in one or more discrete container rotational positions within the well.

23. The biological sample preparation kit of claim 22, wherein the joint keying positions the container such that a hinged container closure opens outward relative to the vibratory agitator head.

24. The biological sample preparation kit of claim 22, wherein a sidewall portion of the well is splayable to provide a friction fit with a tapered body portion of a container received therein.

25. The biological sample preparation kit of claim 22, wherein the keying comprises interference between one or more projections on the container and the well in all but the one or more discreet rotational positions.

26. The biological sample preparation kit of claim 22, wherein the container comprises a plurality of radial projections defining a base about a tapered body portion of the container.

27. The biological sample preparation kit of claim 21, comprising a plurality of wells defined on the vibratory agitator head and keyed to accommodate a plurality of containers with respective hinged closures in a fully open position without interference with access to an adjacent one of the plurality of containers.

28. The biological sample preparation kit of claim 21, wherein at least one of the container and the well define a self-centering taper.

29. The biological sample preparation kit of claim 21, further comprising a plurality of containers, a plurality of handles, a plurality of elongate probes, a plurality of glass slides, and one or more liquid reservoirs.

30. The biological sample preparation kit of claim 29, further comprising a vacuum pen for indirect handling of the glass slides.

31. A biological sample preparation kit comprising:
a container for receiving, a biological, sample and a liquid;
a handle defining an exterior grip portion and an interior recessed screen portion defining tapered apertures;
an elongated probe attachable to the handle to extend therefrom to collect a biological sample;
wherein the handle is removably couplable to the container with the elongated probe received within the container, and the elongated probe is separable from the handle.

32. The biological sample preparation kit of claim 3 wherein the elongated probe is removably retained at one end by the handle.

33. The biological sample preparation kit of claim 31, wherein the elongated probe is secured within a recess in the handle via at least one of a snap fit, interference fit, and threaded fit.

34. The biological sample preparation kit of claim 31, wherein the plurality of the apertures are downwardly tapered to minimize horizontal surface area presented to floatation of sample constituents.

35. The biological sample preparation kit of claim 31, wherein the handle further defines a spillover recess between the grip and the screen.
